(19) United States
(12) Patent Application Publication
(10) Pub. No.: US 2005/0106262 A1
(43) Pub. Date: May 19, 2005

(54) Pharmaceutical composition comprising arsenite for the treatment of malignancy

(75) Inventors: Sang Bong Lee, Anyang City (KR); Yong Jin Yang, Seongnam City (KR)

Correspondence Address: PEACOCK MYERS AND ADAMS P C, P O BOX 26927, ALBUQUERQUE, NM, 871256927, US

(73) Assignee: Korea Microbiological Laboratories, Ltd., Shihung City (KR)

(21) Appl. No.: 10/962,357

(22) Filed: Oct. 8, 2004

Related U.S. Application Data
(63) Continuation of application No. PCT/NL02/00231, filed on Apr. 10, 2002.

(51) Int. Cl. A61K 33/36

(52) U.S. Cl. 424/623

(57) ABSTRACT

A pharmaceutical composition for the treatment of a solid malignancy chosen from the group consisting of colon tumour, gastric tumour, mammary tumour, ovarian tumour, prostate tumour, and renal tumour, said composition comprising a salt of meta-arsenite (AsO₂⁻) and a pharmaceutically acceptable auxiliary. The invention also relates to its use and to a method of treatment.

The present invention relates to a pharmaceutical composition containing arsenic for the treatment of a malignancy.

Pharmaceutical compositions containing arsenic are known for cancer therapy. For example, the review by Waxman S. et al (The Oncologist 6(suppl. 2), pp. 3-10 (2001)) describes arsenic disulfide, arsenic trisulfide and arsenic trioxide the use of said composition comprising a salt of meta-arsenite (AsO₂⁻) and a pharmaceutically acceptable auxiliary.

Due to their inherent toxicity, and the advent of good alternatives, interest in arsenic compounds has remained low.

The object of the present invention is to provide a pharmaceutical composition suitable for use in cancer therapy, which allows for the treatment of solid tumours. The pharmaceutical composition may be used for treatment of such solid tumours for which currently no treatment exists, or as an alternative or supplementary treatment for such solid tumours.

To this end, the pharmaceutical composition according to the present invention is characterized in that it is a pharmaceutical composition for the treatment of a solid malignancy chosen from the group consisting of colon tumour, gastric tumour, mammary tumour, ovarian tumour, prostate tumour, and renal tumour, said composition comprising a salt of meta-arsenite (AsO₂⁻) and a pharmaceutically acceptable auxiliary.

It has been found that the specified types of tumour are surprisingly sensitive to the meta-arsenite salt. In the salt the counter-ion of meta-arsenite may be any pharmaceutically acceptable counter-ion.

In the article by Waxman, mention is made of an article by Tarnowski G. S. et al (Cancer Research, 26(2), pp. 181-206 (1966)) where 8 tumour types were investigated with 14 different anti-tumour chemicals. Potassium arsenite affected only the growth of Ehrlich ascites tumour. Regarding this tumour type it is remarked that it is more sensitive in the ascites than in the sold form. This emphasizes the surprising finding of the present invention.

According to a preferred embodiment, the salt is an alkaline or earth alkaline metal salt. According to a more preferred embodiment, the alkaline metal salt is a potassium or sodium salt.

Such salts are readily soluble and are readily available for exerting their anti-tumour effect.

The invention also relates to the use of a salt of meta-arsenite (AsO₂⁻) for the manufacture of a pharmaceutical composition for the treatment of a solid malignancy chosen from the group consisting of colon tumour, gastric tumour, mammary tumour, ovarian tumour, prostate tumour, and renal tumour.

It was found that solid malignancy belonging to the group consisting of colon tumour, mammary tumour, prostate tumour, and renal tumour were particularly sensitive.

Finally, the present invention relates to a method of treating a human individual suffering from a solid malignancy chosen from the group consisting of colon tumour, gastric tumour, mammary tumour, ovarian tumour, prostate tumour, and renal tumour, with a pharmaceutically effective dose of a salt of meta-arsenite (AsO₂⁻).

The present invention will now be elucidated with reference to the following non-limiting example.

### EXAMPLE

Various human tumor cells were grown at 37° C. in a humidified atmosphere (95% air, 5% CO₂) in monolayer cultures in RPMI 1640 medium with phenol red (Life Technologies, Karlsruhe, Germany) supplemented with 10% fetal calf serum. Cells were trypsinized and maintained weekly.

Cytotoxicity Assay

A modified propidium iodide assay (based on W. A. Dengler et. al, Anti-Cancer Drugs, 6, pp. 522-532 (1995)) was used to examine the antiproliferative activity of the study compounds. Briefly, cells will be harvested from exponential phase cultures growing in RPMI 1640 medium supplemented with 10% fetal calf serum by trypsination, counted and plated in 96 well flat-bottomed microtiter plates (140 μl cell suspension, 8×10⁴ cells/ml). After a 24 h recovery, to allow cells to resume exponential growth, 10 μl culture medium (6 control wells per plate) or culture medium containing the test drug were added to the wells. Each drug concentration was plated in triplicate. After 4 days of incubation culture medium was replaced by an aqueous propidium iodide solution (6 μg/ml). Microtiter plates were kept at −18° C. for 24 h, resulting in a total cell kill. After thawing of the plates, fluorescence was measured using a Millipore Cytofluor 2350-microplate reader (excitation 530 nm, emission 620 nm) in order to quantify the total cell number. The assay included untreated and positive controls (5-FU and vindesine).

Growth inhibition is expressed as Treated/Control×100 (or T/C %). IC₅₀ and IC₇₀ values were determined by plotting compound concentration versus cell number. Mean IC₅₀ and IC₇₀ values were calculated according to the formula:

$$Mean {IC}_{50 , 70} = \frac{{\sum }_{x = 1}^{n} {log ( {IC}_{50 , 70} )}_{x}}{{10}^{n}}$$

With x=specific tumor cell line and n=total number of cell lines studied. If IC₅₀ or IC₇₀ could not be determined within the examined dose range, the lowest or highest concentration studied was used for the calculation.

Assays were considered valid only if the positive control (5-FU) induced a tumor growth inhibition of T/C<30% and if vehicle treated control cells had a fluorescence intensity>500 units.

Results

The results have been summarized in Table I, which shows that in particular tumour cell lines of the type gastric tumour, ovarian tumour, and in particular prostate tumour, mammary tumour, renal and colon tumour were sensitive to the meta-arsenite compound. In comparison, promyelocytic leukaemia, which is known to respond to arsenic trioxide, showed an IC₇₀ value of 6.82 μg/ml. Hence, the tumour cells to which the present invention relates are about 2 to 20 times more sensitive to the meta-arsenite compound according to the present invention.

TABLE I

| Tumour | cell line | IC₇₀ (μg/ml) |
|---|---|---|
| Colon | DLD1 | 0.48 |
| Gastric | GXF251L | 3.08 |
| Mammary | MXAF401NL | 0.32 |
| Ovarian | OVCAR3 | 3.02 |
| Prostate | PC3 | 0.85 |
| Renal | RXF486L | 0.63 |

1. Pharmaceutical composition for the treatment of a solid malignancy chosen from the group consisting of colon tumour, gastric tumour, mammary tumour, ovarian tumour, prostate tumour, and renal tumour, said composition comprising a salt of meta-arsenite (AsO₂⁻) and a pharmaceutically acceptable auxiliary.

2. Pharmaceutical composition according to claim 1, wherein the salt is an alkaline or earth alkaline metal salt.

3. Pharmaceutical composition according to claim 2, wherein the alkaline metal salt is a potassium or sodium salt.

4. Use of a salt or meta-arsenite (AsO₂⁻) for manufacture of a pharmaceutical composition for the treatment of a solid malignancy chosen from the group consisting of colon tumour, gastric tumour, mammary tumour, ovarian tumour, prostate tumour, and renal tumour.

5. Use according to claim 4, wherein solid malignancy is chosen from the group consisting of colon tumour, gastric tumour, mammary tumour, ovarian tumour, prostate tumour, and renal tumour.

6. Method of treating a human individual suffering from a solid malignancy chosen from the group consisting of colon tumour, gastric tumour, mammary tumour, ovarian tumour, prostate tumour, and renal tumour, by providing and employing a pharmaceutically effective dose of a salt of meta-arsenite (AsO₂⁻).
